(19) United States
(12) Patent Application Publication
(10) Pub. No.: US 2025/0283889 A1
(43) Pub. Date: Sep. 11, 2025

(54) METHOD AND COMPOSITION FOR ASSAYING STEM CELL MIMETIC PEPTIDES IN VITRO

(71) Applicant: GENEWORKS, LLC, Flower Mound, TX (US)

(72) Inventors: Troy WADE ANDERSEN, Flower Mound, TX (US); Bryant Giovanni Valenti, Argyle, TX (US)

(21) Appl. No.: 18/861,530

(22) PCT Filed: Apr. 27, 2023
(86) PCT No.: PCT/US2023/020266

§ 371 Date: Oct. 29, 2024

Related U.S. Application Data
(60) Provisional application No. 63/336,708, filed on Apr. 29, 2022.
(60) Provisional application No. 63/400,071, filed on Aug. 23, 2022.
(60) Provisional application No. 63/400,072, filed on Aug. 23, 2022.

(51) Int. Cl. G01N 33/68; C08J 3/075; C08J 3/24; G01N 21/64

(52) U.S. Cl. CPC G01N 33/6803; C08J 3/075; C08J 3/24; G01N 21/6486; C08J 2389/00

(57) ABSTRACT

A method and kit for preparing a cosmetic spray or hydrogel solution comprising a fluorescent indicator for measuring relative stability and activity of bio-active constituents after reconstitution. The spray solution is prepared by diluting a lyophilized protein mix with a diluent or constitution fluid. The hydrogel or spray solution composition can include a protein mix comprising at least one of a plurality of growth factors including bFGF, EGF, GDF-11, HGF, KGF, PDGF-AA, TGF-b1, and VEGF. The lyophilized protein mix also includes a fluorescent indicator such as GFP or BFP and UV-light such as a blacklight for an end-user to quickly and accurately determine the relative viability and/or activity and stability of other bio-active ingredients in the mix. For example, an end-user would be able to quickly determine whether the bio-active ingredients survived the manufacturing, shipping, and reconstitution process.

This application claims priority to U.S. Provisional App. No. 63/336,708 filed Apr. 29, 2022, for a Hydrogel Composition, Method for Producing the Same, And Use Thereof for Stem Cell Mimetic Peptide Supplementation; U.S. Provisional App. No. 63/400,071 filed Aug. 23, 2022, for a Spray Composition, Method for Producing the Same, And Use Thereof for Stem Cell Mimetic Peptide Supplementation; and U.S. Provisional App. No. 63/400,072 filed Aug. 23, 2022, for a Method and Composition for Assaying Stem Cell Mimetic Peptides In Vitro; and incorporates the same by reference.

### FIELD OF DISCLOSURE

The present disclosure relates to a method and composition for assaying enzymatic activity in vitro and, more specifically, a hydrogel, spray solution, and cosmetic kit comprising proteins and peptides and a fluorescent indicator for assaying enzymatic activity in vitro.

### BACKGROUND OF THE DISCLOSURE

Fluorescence is an emission phenomenon in which the wavelength of emitted (fluoresced) wavelength is always higher than with absorbed radiation (with higher energy). Fluorometric enzyme assays typically use a difference in the fluorescence spectra of substrate from product to measure the enzyme reaction. Uses of fluorescent dyes range from fluorescently tagged antibodies useful for determining protein expression and localization, to reporters attached to sensor motifs (e.g. metal ion, reactive oxygen species, pH) to serving as substrates for a broad variety of enzymatic activities.

Green fluorescent protein (GFP) and blue fluorescent protein (BFP) are proteins that exhibit bright green or blue fluorescence when exposed to light in the blue to ultraviolet range, such as from a blacklight. GFP and BFP are excellent tools in many areas of biology due to their ability to form an internal chromophore without requiring any accessory cofactors, gene products, or enzymes/substrates other than molecular oxygen. The disclosure is not limited by fluorescent protein types, and other fluorescent proteins are envisioned that would serve the purposes of indicated the viability of the proteins in the protein mix, precursor solution, hydrogel, or spray composition.

A major problem currently facing the cosmetics industry and other industries is that it is not possible to determine the stability of certain bio-active ingredients. For example, in a hydrogel or spray cosmetic containing reconstituted enzymes, proteins, and peptides, it is not possible for an end consumer to determine how long after reconstitution the bio-active ingredients remain active.

Hydrogels and spray solutions have been implemented in a wide variety of applications in the treatment and supplementation of biological systems. Their adaptability allows them to be used in a range of human health applications such as for use as a topical agent for treatment of the epidermis.

Often cosmetic applications come with the constituent ingredients pre-dissolved in a solution. As a coating for the epidermis, these sprays serve a variety of functions such as improving the aesthetic appearance of skin or treating certain conditions such as for treating infection from microorganisms. Many of these sprays contain active enzymes or other biological agents which are prone to breaking down and, thus, require careful handling to ensure product stability from the point of manufacture to the end-consumer. For example, many enzymes are required to be kept under ultra-cold conditions to maintain efficacy and prevent degradation. Most currently available sprays and hydrogels advertised as containing active enzymes are sold under conditions that would render the enzymes biologically inactive by the time the enzymes reach the consumer. This is because the active ingredients of these products are less stable in the room-temperature solutions in which they are kept and sold. However, consumers are generally unaware that enzymes can slowly become biologically inactive over time.

Stem cells and peptides have been proven to deliver beneficial effects to human skin. The natural degradation of peptides and other active ingredients in cosmetic treatments prior to application is deleterious to the treatments. Peptides are most resistant to natural degradation when in lyophilized form. Yet current cosmetic products containing stem cells and peptides on the market fail to achieve optimum efficacy because these products fail to protect the stem cell and peptide ingredients from degradation. Additionally, stem cells and peptides require advanced equipment and knowledge in order to maintain the viability of these sensitive agents such as proper storage and handling. Many of the peptides get denatured in the manufacturing process and those that survive are denatured or unravel when left at room temperature for more than a day or two. Thus, by the time these products reach the consumer, much of the peptide and stem cell ingredients are no longer active or have fully denatured.

One problem yet to be solved is the production of a spray solution or hydrogel which can be easily made by a user and which allows for the on-site reconstitution of lyophilized, stabilized enzymes and other anti-aging, skin rejuvenating compounds such as mimetic peptides, thereby avoiding degradation of the active ingredients within the spray solution during shipment to the end-user.

On the market today, there are currently no available freeze-dried cosmetics which can be reconstituted by an end-user to make the constituent ingredients bioactive just prior to application. Currently available cosmetics with bioactive ingredients are shipped with the active ingredients already dissolved in hydrated form, thus, rendering them prone to degradation. These existing products attempt to compensate for the degradation by producing solutions with larger concentrations of bioactive peptides, starting in the milligram range. Significant degradation of the active ingredients is due to the decreased stability of these products in solution. However, even if these products did not experience degradation, the bioactive ingredients and peptides would be dissolved in too high of a concentration to effectively penetrate skin, and due to the high concentrations, can lead to inflammation. Thus, existing cosmetics on the market today fail to deliver bioactive ingredients to an end-user in any amount that would not cause inflammation and would still be therapeutically effective.

The present disclosure solves the problem of premature degradation of the active ingredients prior to application by shipping the bio-active ingredients in lyophilized form for reconstitution by an end-user just prior to application. The present disclosure relates to a spray solution or hydrogel which can dissolve lyophilized picogram and nanogram quantities of stem cell mimetic biomolecules and other anti-aging, skin rejuvenating compounds for on-site reconstitution. The pico-gram and nano-gram concentrations decrease the incidence of inflammation. On-site reconstitution thereby prevents degradation of the bioactive ingredients and ensures dose accuracy which can decrease the incidence of an associated inflammation response. The lyophilized protein mix also includes a fluorescent indicator such as GFP or BFP—although other fluorescent proteins are envisioned and the disclosure is not limited by the type of fluorescent protein, as other fluorescent proteins and indicators are envisioned. Additionally, the disclosure is not limited to UV-light or black light or any particular light frequency. Other suitable frequencies of light which induce fluorescence of fluorescent proteins and indicators are envisioned.

In certain embodiments, a blacklight can be used by an end-user to quickly and accurately determine the relative viability and/or activity and stability of other bio-active ingredients in the mix. For example, an end-user would be able to quickly determine whether the bio-active ingredients survived the manufacturing, shipping, and reconstitution process.

Thus, what is needed is a method for producing a hydrogel or spray composition for mimetic peptide supplementation, the method allowing a user the means for mixing lyophilized mimetic stem cell peptides at a site of use, a fluorescent indicator for measuring relative stability and activity of bio-active constituents after reconstitution, and a kit for applying the same.

### SUMMARY OF THE INVENTION

In consideration of the foregoing, the present disclosure relates to a method of preparing a new and unique spray composition or hydrogel for use in mimetic stem cell peptide supplementation which has improved properties relative to existing spray compositions, methods of making said compositions, and methods for applying and using said compositions. Specifically, the present disclosure relates to a spray composition which includes bio-active components to be reconstituted on-site by a user for topical administration of a mimetic peptide supplement composition, and a fluorescent indicator for measuring relative stability and activity of said bio-active constituents after reconstitution.

In certain aspects, the present disclosure relates to a cosmetic spray or hydrogel composition adapted to deliver a composition of lyophilized peptides that can be stored at room temperature for years and be made bio-active at the time of a customer's choosing. The composition comprises a diluent blend and initiator fluid with a constituted lyophilized peptide mixture into a bio-active liquid or hydrogel state. The present disclosure also relates to a method for preparing the composition to ensure homogeneity in the composition before initiating a chemical reaction to transform a liquid precursor solution into a hydrogel or spray which can be easily applied to the skin. The hydrogel and spray composition comprise a unique combination of components and is especially adapted to create a desirable consistency and viscosity and keep within tolerances so as to not harm bio-active proteins of the spray composition.

Furthermore, the present disclosure relates to a custom broad-spectrum low-dose supplement composition which can be included in the hydrogel or spray, the supplement composition comprising enzymes, proteins, peptides, and skin penetrating peptide(s) for optimum effect in delivering live and bio-active proteins into regions of the skin wherein they are readily available for adsorption to and absorption by skin cells and surrounding tissue. The unique broad-spectrum composition possesses synergistic effects such that smaller doses of the components of the broad-spectrum composition are unexpectedly more efficacious than larger doses of single peptides or narrow-spectrum compositions. As a feature of the present disclosure, the mimetic peptides of the broad-spectrum composition can be bio-conjugated to cell-penetrating peptides for increased penetration of the mimetic peptides at lower dosages. Additionally, the broad-spectrum composition can also be administered with skin permeation enhancers. Additionally, cell penetrating peptide selection can be based on the peptides' attributes as a skin penetrating peptide or skin permeation enhancer.

As another feature of the present disclosure, certain peptides such as umbilical cord stem cells growth factor are selected for their unexpected synergistic effect which enhances the efficacy of broad-spectrum compositions allowing them to be administered at significantly lower concentrations than previously seen.

Another feature of the present disclosure includes Poly Lactic-co-Glycolic Acid (PLGA) as biodegradable controlled drug delivery carrier for the broad-spectrum low-dose recombinant protein composition. The broad-spectrum composition can include PLGA nanospheres bound to the mimetic peptides. After application of the broad-spectrum composition, the nanospheres can be engineered to break down over the course of a predetermined optimal period of time, for example, hours or days, after which a subsequent treatment can be performed. In another embodiment, micro-spicules are included in the composition to allow a user, after topical application, to push the nanospheres into the skin with the growth factors electrostatically adsorbed to the surface of the nanospheres.

As a feature of the present disclosure, in some embodiments the peptides of the broad-spectrum composition can be bio-conjugated to cell-penetrating peptides (CPPs) for increased penetration of the peptides at lower dosages. CPPs are short amino acid sequences that can penetrate cell membranes and deliver various molecules into cells. By way of example, it is envisioned that the following CPPs can be combined with the supplement composition, however, the disclosure is not limited to just the following CPPs and other CPPs are envisioned that are analogous to the following or operate via similar mechanisms: Tat peptide, derived from the HIV-1 Tat protein, this peptide has been extensively used to deliver various cargoes into cells; Antennapedia peptide, derived from the Drosophila Antennapedia homeodomain protein, this peptide has been used for the delivery of a wide range of molecules, including proteins, peptides, and nucleic acids; Transportan, a chimeric peptide composed of a fragment of the neuropeptide galanin and a fragment of the antimicrobial peptide cecropin B, this peptide has been shown to efficiently deliver nucleic acids into cells, which according to some embodiments, the supplement composition may comprise nucleic acid sequences such as DNA or RNA encoding transcription or regulation of peptides; Penetrating, derived from the Drosophila homeodomain protein Antennapedia, this peptide has been shown to deliver a wide range of molecules, including proteins, peptides, and nucleic acids; VP22, derived from the Herpes simplex virus type 1 (HSV-1), this peptide has been shown to efficiently deliver various molecules, including proteins, peptides, and nucleic acids, into cells; R9, a Nona-arginine peptide, this peptide has been shown to efficiently deliver various molecules, including proteins, peptides, and nucleic acids, into cells; PTD-4, a 12-amino acid peptide derived from the transcriptional activator protein of the HIV-1 virus, this peptide has been shown to deliver various molecules, including proteins, peptides, and nucleic acids, into cells; MPG peptide, a chimeric peptide composed of a hydrophobic sequence and a cationic peptide, this peptide has been shown to efficiently deliver nucleic acids into cells; Pep-1, a synthetic peptide composed of 16 amino acids, this peptide has been shown to efficiently deliver various molecules, including proteins, peptides, and nucleic acids, into cells; CADY peptide, a cyclic peptide composed of six amino acids. this peptide has been shown to efficiently deliver siRNA into cells.

Additionally, in some embodiments the broad-spectrum composition can also be administered with skin permeation enhancers (SPEs). SPEs are compounds that are used to enhance the penetration of drugs and other substances through the skin. By way of example, it is envisioned that the following SPEs can be combined with the supplement composition, however, the disclosure is not limited to just the following SPEs and other SPEs are envisioned that are analogous to the following or operate via similar mechanisms: Azone, a polar, nonionic surfactant that can increase the permeability of the skin by disrupting the lipid bilayer structure of the stratum corneum; Oleic acid, a fatty acid that can increase the penetration of lipophilic drugs through the skin by disrupting the lipid matrix of the stratum corneum; Ethanol, a polar solvent that can enhance the permeability of the skin by increasing the fluidity of the stratum corneum and disrupting the intercellular lipid lamellae; Dimethyl sulfoxide (DMSO), a polar solvent that can increase the permeability of the skin by disrupting the lipid bilayer structure of the stratum corneum and increasing the solubility of lipophilic drugs; Sodium lauryl sulfate (SLS), an anionic surfactant that can enhance the penetration of hydrophilic drugs through the skin by disrupting the intercellular lipid lamellae and increasing the solubility of hydrophilic drugs; Menthol, a cyclic terpene that can enhance the penetration of drugs through the skin by disrupting the lipid bilayer structure of the stratum corneum and increasing the solubility of lipophilic drugs; Propylene glycol, a polar solvent that can increase the permeability of the skin by increasing the fluidity of the stratum corneum and disrupting the intercellular lipid lamellae; Urea, a polar compound that can enhance the penetration of drugs through the skin by increasing the water content of the stratum corneum and disrupting the intercellular lipid lamellae; Terpenes, a class of cyclic hydrocarbons that can enhance the penetration of drugs through the skin by disrupting the lipid bilayer structure of the stratum corneum and increasing the solubility of lipophilic drugs; and Phospholipids, a class of lipids that can enhance the penetration of drugs through the skin by integrating into the stratum corneum lipid matrix and increasing the fluidity of the intercellular lipid lamellae.

Additionally, in some embodiments cell penetrating peptide selection can be based on the peptides' attributes as a skin penetrating peptide or skin permeation enhancer. In some embodiments, skin electroporation might also be implemented along with application of the supplement composition to promote uptake of the components of the composition into cells.

As another feature of the present disclosure, according to some embodiment's certain peptides such as stem cell growth factors or umbilical cord stem cells growth factors are selected for their unexpected synergistic effect which enhances the efficacy of broad-spectrum compositions allowing them to be administered at significantly lower concentrations than previously seen.

According to other embodiments, another feature of the present disclosure includes Poly Lactic-co-Glycolic Acid (PLGA) as biodegradable controlled drug delivery carrier for the broad-spectrum low-dose recombinant protein composition. PLGA is a biocompatible polymer that has been extensively used for drug delivery and delivery of biologics and tissue engineering applications due to its unique properties such as tunable degradation rate, controlled drug release, and ability to form porous structures. The disclosure is not limited to PLGA and other polymers that have similar properties to PLGA can suffice as a substitute, including, but not limited to: Poly (ε-caprolactone) (PCL), a biodegradable polyester that degrades more slowly than PLGA and has been used for long-term drug delivery and tissue engineering applications; Poly (lactic acid) (PLA), a biodegradable polymer that degrades more rapidly than PLGA and has been used for drug delivery and tissue engineering applications; Poly (glycolic acid) (PGA), a biodegradable polymer that degrades more rapidly than PLGA and has been used for drug delivery and tissue engineering applications; Poly (d,l-lactide-co-caprolactone) (PLCL), a copolymer of lactide and caprolactone that can be tailored to degrade over a wide range of time periods and has been used for drug delivery and tissue engineering applications; Poly (ethylene glycol) (PEG), a biocompatible polymer that has been used as a carrier for drug delivery and tissue engineering applications due to its ability to prolong the circulation time of drugs and reduce their immunogenicity; Poly (vinyl alcohol) (PVA), a water-soluble polymer that has been used for drug delivery applications due to its ability to form hydrogels and control drug release; Poly (hydroxyethyl methacrylate) (PHEMA), a hydrophilic polymer that has been used for drug delivery and tissue engineering applications due to its ability to form hydrogels and its biocompatibility; Poly (propylene fumarate) (PPF), a cross-linkable polymer that has been used for tissue engineering applications due to its ability to form porous scaffolds and support tissue growth; Poly (anhydride-co-imide) (PAI), a biodegradable polymer that has been used for drug delivery and tissue engineering applications due to its ability to degrade rapidly and form a porous structure; and Poly (caprolactone fumarate) (PCLF), a cross-linkable polymer that has been used for tissue engineering applications due to its ability to form porous scaffolds and support tissue growth.

In certain embodiments, the broad-spectrum supplement composition comprises vitamins. Vitamins are essential organic compounds required by the human body to perform various functions such as growth, development, and maintenance of bodily tissues. There are 13 types of vitamins that can be included which have been classified into two categories: fat-soluble and water-soluble. Fat-soluble vitamins are those that dissolve in fat and are stored in the body's fatty tissues. These include vitamins A, D, E, and K. Vitamin A is important for maintaining healthy vision, skin, and immune system. Vitamin D helps the body absorb calcium and maintain bone health. Vitamin E acts as an antioxidant and protects the body from damage caused by free radicals. Vitamin K is necessary for blood clotting and bone health. Water-soluble vitamins are those that dissolve in water and are not stored in the body for long periods. These include vitamins B1 (thiamine), B2 (riboflavin), B3 (niacin), B5 (pantothenic acid), B6 (pyridoxine), B7 (biotin), B9 (folate), and B12 (cobalamin), as well as vitamin C. The B vitamins are important for energy production, brain function, and maintaining healthy skin, hair, and nails. Vitamin C is an antioxidant that helps protect the body from damage caused by free radicals and is important for immune system function. Overall, vitamins are crucial for maintaining good health, and a balanced and varied diet is the best way to ensure that the body receives all the necessary vitamins and minerals it needs.

In certain embodiments, the supplement composition can include PLGA nanospheres bound to the peptides. After injection of the supplement composition, the nanospheres can be break down over the course of a predetermined optimal period of time for time determinant supplementation, for example, one month, after which a subsequent treatment can be performed. In another embodiment, micro-spicμles are used to push nanospheres into the skin with the proteins, peptides, or growth factors, for example, electrostatically adsorbed to the surface of the nanospheres.

In certain embodiments, the disclosure relates to a spray solution. Spray solutions have been implemented in a wide variety of applications in the treatment and supplementation of biological systems. Their adaptability allows them to be used in a range of human health applications such as for use as a topical agent for treatment of the epidermis.

Most spray solutions employed in cosmetic applications come with the constituent ingredients pre-dissolved in a solution. As a coating for the epidermis, these sprays serve a variety of functions such as improving the aesthetic appearance of skin or treating certain conditions such as for treating infection from microorganisms. Many of these sprays contain active enzymes or other biological agents which are prone to breaking down and, thus, require careful handling to ensure product stability from the point of manufacture to the end-consumer. For example, many enzymes are required to be kept under ultra-cold conditions to maintain efficacy and prevent degradation. Most currently available sprays advertised as containing active enzymes are sold under conditions that would render the enzymes biologically inactive by the time the enzymes reach the consumer.

The present disclosure relates to a spray solution which can deliver lyophilized, stable picogram and nanogram concentrations of stem cell mimetic biomolecules and other anti-aging, skin rejuvenating compounds for on-site dissolution thereby preventing degradation of the bioactive ingredients and ensuring dose accuracy which can decrease the incidence of an associated inflammation response.

In consideration of the foregoing, the present disclosure relates to a method of preparing a new and unique spray composition for use in mimetic stem cell peptide supplementation which has improved properties relative to existing spray compositions, methods of making said compositions, and methods for applying and using said compositions. Specifically, the present disclosure relates to a spray composition which may be produced on-site by a user for topical administration of a mimetic peptide supplement composition.

In certain aspects, the present disclosure relates to a cosmetic spray composition adapted to deliver a composition of lyophilized peptides that can be stored at room temperature for years and be made bio-active at the time of a customer's choosing. The composition comprises a diluent blend and initiator fluid to constitute a lyophilized peptide mixture into a bio-active liquid state. The present disclosure also relates to a method for preparing the composition to ensure homogeneity in the composition before initiating a chemical reaction to transform a liquid precursor solution into a spray which can be easily applied to the skin. The spray composition comprises a unique combination of components and is especially adapted to create a desirable consistency and viscosity and keep within tolerances so as to not harm bio-active proteins of the spray composition.

Furthermore, the present disclosure relates to a custom broad-spectrum low-dose protein composition which can be included in the spray, the recombinant proteins being linked to skin penetrating peptide(s) for optimum effect in delivering live and bio-active proteins into regions of the skin wherein they are readily available for adsorption to and absorption by skin cells and surrounding tissue. The unique broad-spectrum composition possesses synergistic effects such that smaller doses of the components of the broad-spectrum composition are unexpectedly more efficacious than larger doses of single peptides or narrow-spectrum compositions. As a feature of the present disclosure, the mimetic peptides of the broad-spectrum composition be bioconjugated to cell-penetrating peptides for increased penetration of the mimetic peptides at lower dosages. Additionally, the broad-spectrum composition can also be administered with skin permeation enhancers. Additionally, cell penetrating peptide selection can be based on the peptides' attributes as a skin penetrating peptide or skin permeation enhancer.

As another feature of the present disclosure, certain peptides such as umbilical cord stem cells growth factor are selected for their unexpected synergistic effect which enhances the efficacy of broad-spectrum compositions allowing them to be administered at significantly lower concentrations than previously seen.

As another feature of the present disclosure, the kit will include a means for determining the enzymatic activity and viability of bioactive ingredients, for example, those dissolved in precursor solution, hydrogel, or the spray solution. In one embodiment, it is envisioned that the spray solution can include a modified fluorescent protein in sufficient concentration to fluoresce upon application of UV light. After constituting the freeze-dried proteins into the spray solution, precursor solution, or hydrogel, the kit will comprise a UV light specially calibrated to induce a fluorescence of the specific concentration of the modified fluorescent protein. If the product glows at a certain level upon shining the UV light on the spray solution, precursory solution, or hydrogel, the customer will know that the product is fresh, bioactive, and ready to use. The level of fluorescence can be used to predict a corresponding enzymatic activity and viability of bioactive ingredients. Failure to glow or a diminished glow upon application of UV light would be an indication that the bioactive ingredients have de-natured and are less viable as compared to a fresh product. Other means for determining the enzymatic activity and viability of bioactive ingredients are also envisioned and considered part of the disclosure.

As another feature of the present disclosure, it is envisioned that the kit further comprises a uniquely designed atomizer cap attached to a vial of lyophilized constituents of the intended spray solution, the atomizer cap comprising a syringe injection port disposed through a top of the atomizer lid, whereby a customer can deliver a special viscosity blended constitution fluid in a clean and sealed environment to activate the lyophilized material inside.

The present disclosure relates to a method and kit for diluting a lyophilized protein mix with a special viscosity blended constitution fluid by injecting said constitution fluid through an injection port of an atomizer cap, thereby creating a spray solution. The method according to the disclosure can be optimally performed with a kit comprising at least one vial comprising lyophilized constituents and at least one syringe comprising constitution fluid. The kit further comprises a uniquely designed atomizer cap attached to the vial of lyophilized constituents, the atomizer cap comprising a syringe injection port disposed through a top of the atomizer lid, whereby a customer can deliver the constitution fluid in a clean and sealed environment to activate the lyophilized constituents inside.

The disclosure further relates to a spray solution composition comprising constitution fluid and a broad-spectrum supplement composition. It is envisioned that the broad-spectrum supplement composition can contain a plurality of anti-aging and skin-rejuvenating agents, compounds, peptides, growth factors, and additives. In certain embodiments, the broad-spectrum supplement composition comprises at least one growth factor selected from the group consisting of beg, EGF, GDF-11, HGF, KGF, PDGF-AA, TGF-b1, and VEGF. In other embodiments, the spray solution further comprises PLGA nanospheres, wherein the growth factors can be adsorbed onto the nanospheres for sustained release from the spray solution. In some preferred embodiments, the PLGA nanospheres arc approximately 100 to 300 nm.

It is envisioned that any one component of the spray solution can be used in the hydrogel or precursor solution and any one component the constituents of the hydrogel can be used in spray solution.

Therefore, based on the foregoing and continuing description, the subject invention in its various embodiments may comprise one or more of the following features in any non-mutually-exclusive combination:
A method of preparing a hydrogel comprising the step of mixing a diluent mix with a lyophilized protein mix to make a precursor solution;
A method of preparing a mixing a diluent mix with a lyophilized protein mix to make a precursor solution, the lyophilized protein mix comprising at least one fluorescent protein and at least one other protein.
A method of preparing a hydrogel comprising the step of adding an initiator mix to the precursor solution to make a hydrogel, the initiator mix inducing an increase in viscosity;
A method of preparing a hydrogel comprising the step of mixing the precursor solution in a vessel before adding the initiator mix.
A method of preparing a hydrogel wherein the diluent mix comprises a carbomer.
A method of preparing a hydrogel wherein the precursor solution further comprises an amino acid stabilizer, humectant, preservative, emollient, and anti-aging ingredients.
A method of preparing a hydrogel wherein the precursor solution further comprises an amino acid stabilizer.
A method of preparing a hydrogel wherein the precursor solution further comprises a humectant.
A method of preparing a hydrogel wherein the precursor solution further comprises a preservative.
A method of preparing a hydrogel wherein the precursor solution further comprises an emollient.
A method of preparing a hydrogel wherein the precursor solution further comprises an anti-aging molecule, compound, protein, or peptide.
A method of preparing a hydrogel wherein the initiator mix is in low viscosity, un-neutralized form.
A method of preparing a hydrogel wherein the initiator mix comprises a pH modifier, a photoinitiator, and a free radical initiator;
A method of preparing a hydrogel wherein the initiator mix comprises a pH modifier.
A method of preparing a hydrogel wherein the initiator mix comprises a photoinitiator.
A method of preparing a hydrogel wherein the initiator mix comprises a free radical initiator.
A method of preparing a hydrogel wherein the initiator mix is introduced at a percentage comprising 0.01 to 5%,
A method of preparing a hydrogel wherein the initiator mix is introduced at a percentage 1%.
A method of preparing a hydrogel wherein wherein the initiator mix comprises at least one pH modifier selected from the group consisting of triethanolamine, NaOH, KOH, and sodium bicarbonate.
A method of preparing a hydrogel comprising injecting a first syringe filled with diluent mix into a first vial comprising lyophilized protein mix, mechanically mixing contents of the first vial, and pulling precursor solution back into the first syringe;
A method of preparing a hydrogel comprising pulling initiator mix into a second syringe and connecting the first syringe to the second syringe via a syringe-to-syringe adapter, and then mechanically mixing contents of the first and second syringes to form a hydrogel;
A method of preparing a hydrogel wherein the carbomer is present in the hydrogel from 0.1 to 1%;
A method of preparing a hydrogel wherein the carbomer is present in the hydrogel from 0.2% to 0.5%;
A method of preparing a hydrogel wherein the hydrogel comprises a carbomer with a percentage of approximately 0.5%; and a pH initiator with a strength of at least 50% and making up 1% of the hydrogel volume.
A method of preparing a hydrogel wherein the amino acid stabilizer is glycine.
A method of preparing a hydrogel wherein the amino acid stabilizer is included in the hydrogel in a preferable concentration of between 5 mg/ml to 30 mg/ml.
A hydrogel precursor comprising a precursor solution comprising a protein mix, the protein mix comprising at least one growth factor selected from the group consisting of bFGF, EGF, GDF-11, HGF, KGF, PDGF-AA, TGF-b1, and VEGF;
A hydrogel precursor comprising a precursor solution wherein the protein mix comprising at least two growth factors selected from the group consisting of bFGF, EGF, GDF-11, HGF, KGF, PDGF-AA, TGF-b1, and VEGF.
A hydrogel precursor comprising a precursor solution wherein the protein mix comprising at least three growth factors selected from the group consisting of bFGF, EGF, GDF-11, HGF, KGF, PDGF-AA, TGF-b1, and VEGF.
A hydrogel precursor comprising a precursor solution wherein the protein mix comprising at least four growth factors selected from the group consisting of bFGF, EGF, GDF-11, HGF, KGF, PDGF-AA, TGF-b1, and VEGF.
A hydrogel precursor comprising a precursor solution wherein the protein mix comprising at least five growth factors selected from the group consisting of bFGF, EGF, GDF-11, HGF, KGF, PDGF-AA, TGF-b1, and VEGF.
A hydrogel precursor comprising a precursor solution wherein the protein mix comprising at least six growth factors selected from the group consisting of bFGF, EGF, GDF-11, HGF, KGF, PDGF-AA, TGF-b1, and VEGF.
A hydrogel precursor comprising a precursor solution wherein the protein mix comprising at least seven growth factors selected from the group consisting of bFGF, EGF, GDF-11, HGF, KGF, PDGF-AA, TGF-b1, and VEGF.
A hydrogel precursor comprising a precursor solution wherein the protein mix comprises the following growth factors: bFGF, EGF, GDF-11, HGF, KGF, PDGF-AA, TGF-b1, and VEGF.
A precursor solution comprising an additive.
A precursor solution comprising a fluorescent protein.
A precursor solution comprising an amino acid stabilizer.
A precursor solution comprising a protein stabilizer.
A precursor solution comprising a humectant.
A precursor solution comprising a preservative.
A precursor solution comprising an emollient.
A precursor solution comprising an anti-aging molecule.
A precursor solution comprising an anti-aging protein.
A precursor solution wherein the hydrogel further comprises a PLGA nanospheres, wherein the growth factors are adsorbed onto the nanospheres for sustained release.
A precursor solution wherein the PLGA nanospheres are approximately 100 to 300 nm.
A precursor solution further comprising micro-spicule.
A precursor solution wherein the broad-spectrum supplement composition further comprises cell penetrating peptide linkers bound to the growth factors.
A precursor solution wherein growth factors are present in the hydrogel composition at a ratio as follows:
   bFGF is present at a ratio of approximately 250.00;
   EGF is present at a ratio of approximately 250.00;
   GDF-11 is present a ratio of approximately 5.00;
   HGF is present at a ratio of approximately 20.00;
   KGF is present at a ratio of approximately 5.00;
   PDGF-AA is present at a ratio of approximately 20.00;
   TGF-b1 is present at a ratio of approximately 10.00; and
   VEGF is present at a ratio of approximately 7.00.
A kit for making a hydrogel comprising at least one vessel comprising lyophilized protein mix;
A kit for making a hydrogel comprising at least one vessel comprising lyophilized protein mix comprising at least one fluorescent protein.
A kit for making a hydrogel comprising at least one vessel comprising diluent mix;
A kit for making a hydrogel comprising at least one vessel comprising initiator mix;
A kit for making a hydrogel wherein the vessels are vials.
A kit for making a hydrogel comprising a first Luer-lock syringe;
A kit for making a hydrogel comprising a second Luer-lock syringe;
A kit for making a hydrogel comprising a syringe-to-syringe connector to connect syringes;
A kit for making a hydrogel comprising a vial-to-syringe adaptor to connect vials with syringes;
A kit for making a hydrogel comprising a black light to illuminate fluorescent protein;
A kit for making a hydrogel wherein, the vessel comprising lyophilized protein mix is a first vial;
A kit for making a hydrogel wherein the vessel comprising diluent mix is a second vial;
A kit for making a hydrogel wherein the vessel comprising initiator mix is a third vial.

### BRIEF DESCRIPTION OF THE FIGURES

FIG. 1 shows a perspective view of a syringe with a Luer-lock adapter.

FIG. 2 shows a perspective view of a black light.

FIG. 3 shows a vessel containing a lyophilized protein mix in the form of a pellet.

FIG. 4 shows a syringe-to-syringe connector/adapter.

FIG. 5 shows a syringe-to-vial connector/adapter.

FIG. 6 shows a vessel comprising diluent mix.

FIG. 7 shows a vessel comprising initiator mix.

FIG. 8 shows two syringes connected with a syringe-to-syringe connector, the syringe on the left filled with hydrogel.

FIG. 9 shows a vessel comprising diluent mix with a syringe-to-vial connector/adapter attached.

FIG. 10 shows a vessel comprising initiator mix with a syringe-to-vial connector/adapter attached.

FIG. 11 shows a vessel comprising initiator mix with a syringe-to-vial connector/adapter attached to the vial and a Luer-lock syringe attached to the syringe-to-vial connector.

FIG. 12 shows a vessel comprising initiator mix with a syringe-to-vial connector/adapter attached to the vial and a Luer-lock syringe attached to the syringe-to-vial connector.

FIG. 13 shows a syringe comprising hydrogel with homogenously dissolved lyophilized protein mix. The syringe is being illuminated with a blacklight which is causing fluorescence of fluorescent proteins from the lyophilized protein mix dissolved in the hydrogel.

FIG. 14 shows the components one embodiment of the hydrogel kit including a vial comprising diluent mix, a vial comprising lyophilized protein mix, a vile comprising initiator mix, two Luer-lock syringes, one syringe-to-syringe adapter, three syringe-to-vial adapters, and a blacklight.

FIG. 15 shows the components of another embodiments of the kit including twenty-nine vials comprising lyophilized protein mix, three vials comprising diluent mix, and three vials comprising initiator mix.

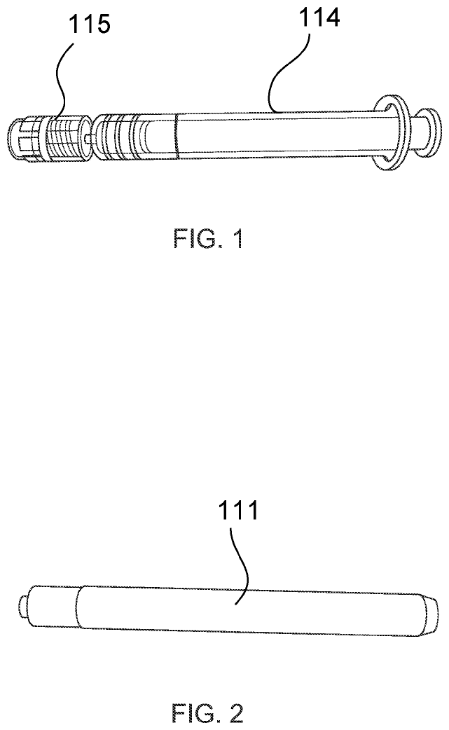

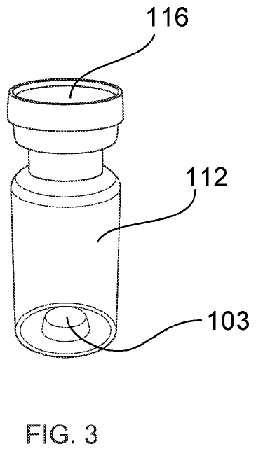

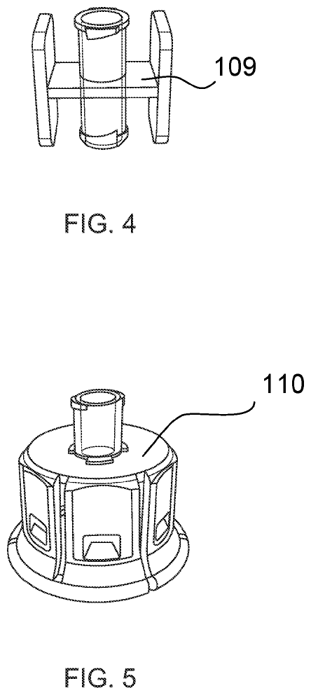

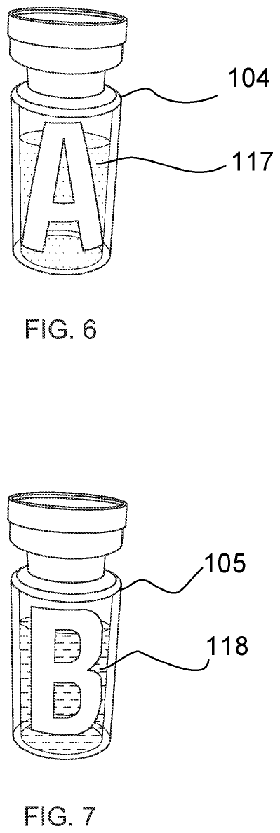

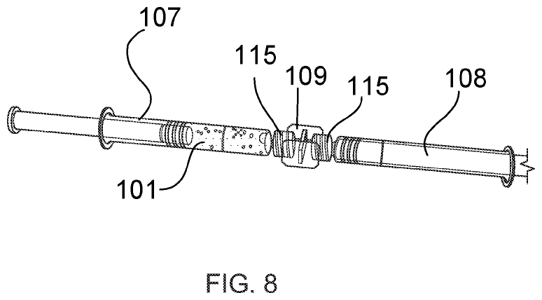

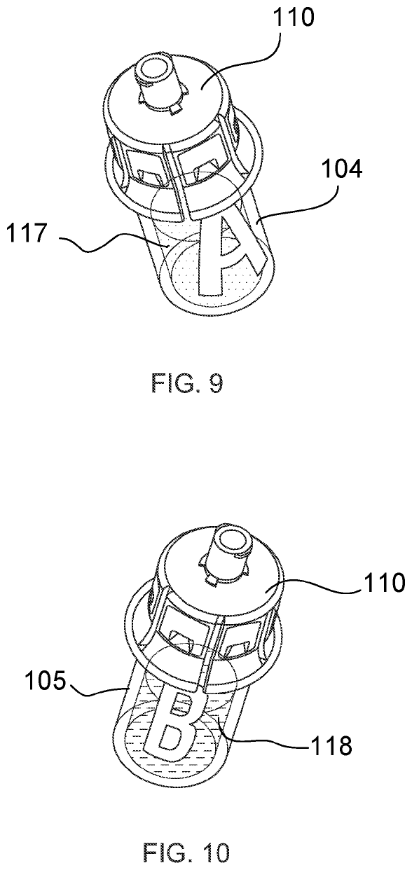

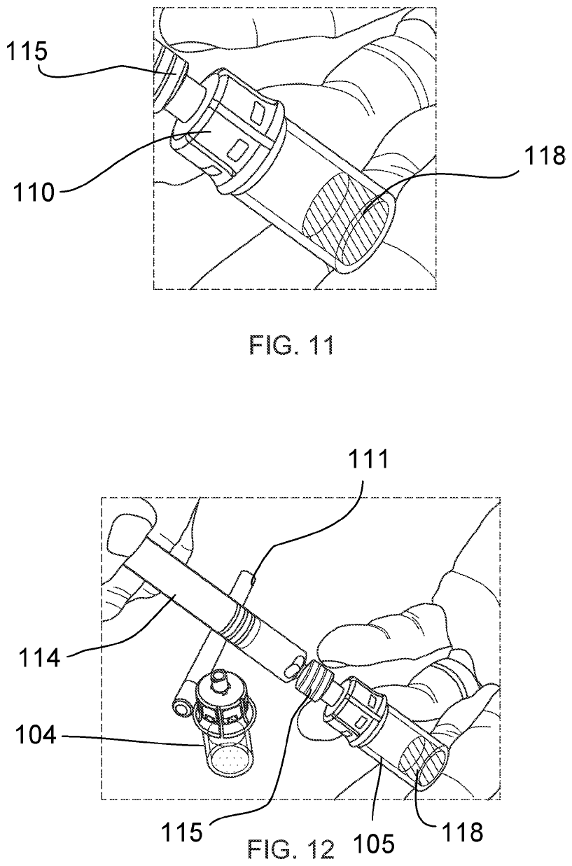

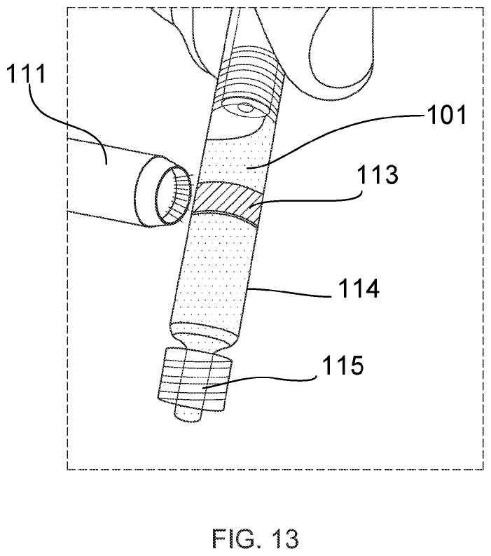

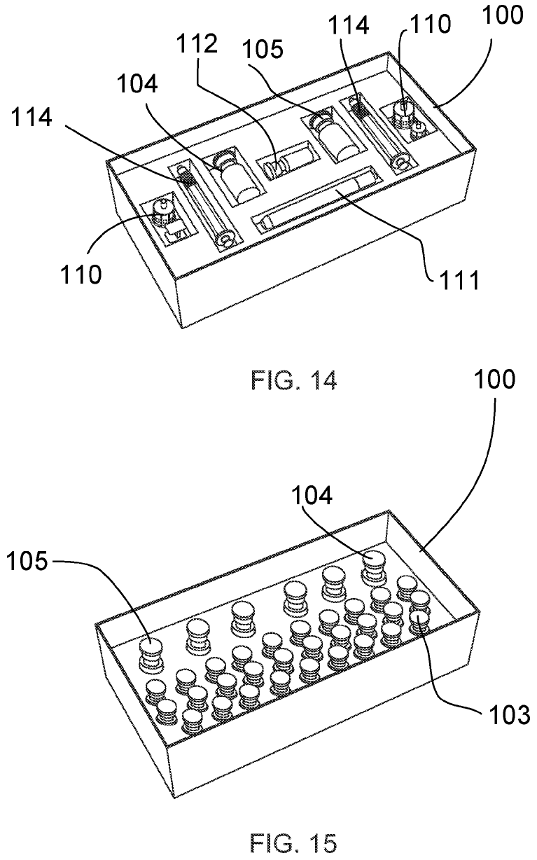

### DETAILED DESCRIPTION OF THE EMBODIMENTS

The present disclosure relates to a method of diluting a lyophilized protein mix with a diluent consisting of an initially low viscosity diluent and inducing a viscosity increase at a future time by inducing a state change by introducing a state change initiator to create a hydrogel with freshly bioactive proteins and peptides. Specifically, the method includes a fluorescent protein indicator as a fresh test for determining the viability of other bio-active proteins in solution or in the hydrogel.

Fluorescent proteins are a type of protein that can emit light when excited by specific wavelengths of light, such as ultraviolet (UV) light. These proteins can be used as indicators of various biological processes. In the case of assessing viability proteins in solution, a fluorescent protein can be used as a reporter protein that is included in the lyophilized protein mix along with other proteins of interest. The reporter protein can then be monitored under light to determine whether the fluorescent protein is viable in that fluoresces upon excitation by light. Fluorescence of the reporter protein can give an indication of whether other proteins of interest in the protein mix are functioning properly. A poor fluorescent result would indicate that the proteins in the mix were not viable. A strong fluorescent result would indicate the freshness and viability of the proteins in the protein mix.

In process, the fluorescent protein and proteins of interest are together in the lyophilized protein mix. After addition of diluent to form a precursor solution, the sample can then be exposed to light, which will cause the fluorescent protein to emit light if it is properly folded and functional. Alternatively, light can be applied after formation of the hydrogel. The fluorescence will give an indication of whether the other proteins of interest are also properly folded and functional.

However, if fluorescent protein is not properly folded or is non-functional, it will not fluoresce, indicating that the other proteins in the mix are also likely not properly folded or are non-functional. This can be used as an indicator that the proteins of interest are not viable or active in the sample.

In certain embodiments, the disclosure relates to a method of preparing a hydrogel comprising the sequential steps of: (step 1) mixing a low viscosity diluent mix with a lyophilized protein mix to make a precursor solution; and (step 2) adding an initiator mix to the precursor solution to make a hydrogel by inducing an increase in viscosity. To promote homogeneity of the precursor solution and resulting hydrogel, it is envisioned that a step 1(a) can be performed after step 1 and before step 2, by mechanically mixing the precursor solution and removing it into an application syringe/vessel before adding the initiator mix. In certain embodiments it is envisioned that the diluent mix comprises a carbomer to act as a thickening agent, emulsifier, or stabilizers.

As used herein, the initiator mix is a combination of chemicals that are used to initiate or start a chemical reaction. In this context, the initiator mix can be used to initiate polymerization reactions in order to create the hydrogel or other products with specific physical and chemical properties. The initiator mix typically comprises at least one activator, which is a chemical that triggers the polymerization reaction, and may also include other additives like stabilizers, accelerators, and inhibitors.

According to the disclosure, the initiator mix comprises at least one activator selected from the group consisting of a pH modifier, a photoinitiator, and a free radical initiator. A pH modifier can be used to adjust the acidity or alkalinity of the mixture, which can affect the rate and extent of the polymerization reaction. A photoinitiator is a chemical that can be activated by exposure to light, and can be used to initiate the reaction in the presence of a light source. A free radical initiator is a chemical that can generate free radicals, which can initiate the polymerization reaction. By using a combination of these activators in the initiator mix, the polymerization reaction can be finely controlled and optimized to achieve the desired properties in the final product. Therefore, in some embodiments, the initiator mix can comprise various combinations of activators.

In some embodiments, the disclosure relates to a hydrogel precursor. As used herein, the hydrogel precursor comprises the precursor solution as well as other additives. The precursor solution can be made according to step 1. Additional steps after step can include the addition of additives, proteins, peptides and other supplement components described herein. Thus, these additional components can be added to the precursor solution to make up the hydrogel precursor.

Low viscosity as used herein refers to the property of a substance that flows easily and smoothly, often characterized by its thin and watery consistency. Viscosity is a measure of a fluid's resistance to flow, with low viscosity indicating that the fluid flows easily and quickly, while high viscosity indicates that the fluid is thick and flows more slowly. Examples of substances with low viscosity include water, alcohol, and many types of oils. Low viscosity is an important characteristic in many applications, such as in the production of coatings, adhesives, and personal care products, where it can help to improve flowability and ease of application. Here, low viscosity of the diluent and precursor solution can be important for promoting homogeneity of mixtures prior to hydrogel formation, which is essential for forming a consistently homogenized hydrogel.

Other biocompatible gel-forming agents that are safe for human application are also envisioned and the disclosure is not intended to be limited by the type of gel-forming agent. For example, Carbomer 940 has a high molecular weight polymer with a high level of cross-linking, which allows it to form a strong gel structure with excellent clarity and stability. Carbomer 940 can also absorb up to 100 times its weight in water making it ideal as an effective thickener and stabilizer.

In addition to Carbomer 940, other carbomers such as Carbomer 934 and Carbomer 941 can also be used for gel formation, depending on the specific application and desired properties of the gel. Carbomer 934 has a lower molecular weight and lower viscosity than Carbomer 940, which makes it more suitable for low viscosity gels. Under certain embodiments, it is envisioned that carbomer 934 is preferred under the disclosure where a low viscosity gel is desired, for example, in application to a large surface area of skin. Carbomer 941 has a higher molecular weight and viscosity than Carbomer 940, therefore, under certain embodiments, it is envisioned that carbomer 941 is more suitable for high viscosity gels, for example, for applications to the face or near the eyes where “melting” of the gel into such areas could cause undesirable effects such as pain.

In certain embodiments, the precursor solution can further comprise at least one additive such as an amino acid stabilizer. Amino acid stabilizers can improve the stability and shelf-life of the composition. Glycine, alanine, proline, glutamic acid, and aspartic acid can be used as stabilizers for proteins and enzymes, while arginine, lysine, histidine, and tyrosine can be used to stabilize other biomolecules such as DNA and RNA. Amino acid stabilizers work by forming hydrogen bonds, electrostatic interactions, and other non-covalent interactions with the target molecules, which helps to prevent denaturation and degradation. They can also improve the solubility and bioavailability of drugs and other active ingredients.

In certain embodiments, the precursor solution can further comprise at least one protein stabilizer. Protein stabilizers can help to maintain the stability, activity, and bioavailability of protein-based drugs. These stabilizers can be classified into several categories, including sugars, polyols, amino acids, surfactants, and salts. Sugars such as trehalose, sucrose, and mannitol can be used as protein stabilizers to protect proteins from denaturation and aggregation under stress conditions such as freeze-drying or high-temperature processing. Polyols such as sorbitol and glycerol can also stabilize proteins by reducing the amount of free water molecules in the formulation, which can promote protein aggregation. Amino acids such as glycine and arginine can form hydrogen bonds and electrostatic interactions with proteins, which can improve their solubility and prevent aggregation. Surfactants such as polysorbate 20 and 80 can also stabilize proteins by forming a protective layer around them, which can reduce their exposure to destabilizing forces. Finally, salts such as sodium chloride and potassium phosphate can stabilize proteins by screening out electrostatic repulsions and promoting favorable interactions.

In certain embodiments, the precursor solution can further comprise at least one additive such as a humectant. Humectants attract and retain moisture, helping to hydrate and soften skin. In certain embodiments, the humectants include glycerin, hyaluronic acid, propylene glycol, urea, and sorbitol. In addition to their moisturizing properties, humectants can also improve the solubility and stability of other ingredients, enhancing the spread ability of products, and reducing the tackiness of certain formulations.

In certain embodiments, the precursor solution can further comprise at least one additive such as a preservative. Preservatives can prevent the growth of harmful microorganisms and ensure the safety and stability of the final product. In certain embodiments, the preservatives include, but are not limited to: Parabens, effective against a wide range of microorganisms and are relatively inexpensive; Phenoxyethanol, a glycol ether that can be effective against bacteria, fungi, and viruses and has low toxicity; Ethylhexylglycerin, a glyceryl ether with antimicrobial; Benzyl alcohol, a naturally occurring alcohol effective against bacteria and fungi and has low toxicity; Sodium benzoate, a salt of benzoic acid that is effective against bacteria, fungi, and yeasts; Potassium sorbate, a salt of sorbic acid that is effective against yeasts and molds.

In certain embodiments, the precursor solution can further comprise at least one additive such as an emollient. Emollients can improve the texture, softness, and smoothness of skin by forming a protective barrier on the skin's surface, preventing moisture loss and keeping the skin hydrated. In certain embodiments, the following emollients can be used based on their unique properties: Natural Oils, such as jojoba, coconut, and olive oil, are rich in fatty acids and have a high affinity for the skin, making them effective at hydrating and softening the skin; Silicones, synthetic compounds that have a silky texture, can provide a smooth and even application, and reduce water loss from the skin; Shea Butter, a natural emollient is rich in fatty acids and has excellent moisturizing properties; Glycerin, a humectant that attracts water to the skin's surface, which can improve the skin's hydration levels; and Lanolin, a natural emollient that is rich in fatty acids and can soothe and hydrate dry, chapped skin.

In certain embodiments, the precursor solution can further comprise at least one additive such as an anti-aging molecule or compound. There are several types of molecules and compounds that can be used in certain embodiments to promote a more youthful appearance, including but not limited to: Retinoids, such as retinol and tretinoin, derived from vitamin A and stimulate collagen production and cell turnover, which can reduce the appearance of fine lines and wrinkles; Peptides, short chains of amino acids that can stimulate collagen production and improve the elasticity of the skin; Antioxidants, such as vitamin C, vitamin E, and green tea extract, can protect the skin from damage caused by free radicals and environmental stressors; Alpha Hydroxy Acids (AHAs), such as glycolic acid and lactic acid, can exfoliate the skin and stimulate cell turnover, which can reduce the appearance of fine lines and wrinkles and Hyaluronic Acid, a humectant that can attract and retain water in the skin, improve hydration levels, and reduce the appearance of fine lines and wrinkles.

In certain embodiments, the precursor solution can further comprise at least one additive such as an anti-aging protein. In certain embodiments, anti-aging proteins can include: Collagen, a fibrous protein responsible for providing structure and support to the skin; Elastin, a protein that can give skin improved elasticity and bounce; Keratin, a protein that is found in the outer layer of the skin, hair, and nails and can protect the skin from environmental damage and maintain its barrier function and strengthen and protect the skin from damage caused by free radicals and other environmental stressors; and Hydrolyzed proteins, proteins that have been broken down into smaller fragments, making them easier to absorb into the skin.

These additives can not only provide stabilization of the peptides and preservation of the hydrogel, but they can also include additional anti-aging ingredients and other active components that promote synergistic effects when combined with growth factor peptides.

In certain embodiments, the amino acid stabilizer is glycine, however, other amino acid stabilizers are envisioned and suitable and the disclosure is not intended to be limited by the type of amino acid stabilizer. In some embodiments, the amino acid stabilizer is included in the hydrogel in a preferable concentration range of between 1 mg/ml to 30 mg/ml and more preferably, 5 mg/ml to 30 mg/ml.

In some embodiments, the hydrogel comprises a carbomer with a percentage of approximately 0.5% and a pH initiator with a strength of at least 50% and making up 1% of the hydrogel volume. In other embodiments, the carbomer is present in the hydrogel from 0.1 to 1%, and preferably 0.2% to 0.5%.

It is envisioned that the initiator mix can be in low viscosity, unneutralized form. In some embodiments, the initiator mix can comprise at least one activator selected from the group consisting of a pH modifier, a photoinitiator, and a free radical initiator. In some preferred embodiments, the initiator mix is introduced at a percentage range of 0.01 to 5%, and preferably 1%. In some embodiments, it is envisioned that the pH modifier can be selected from the group consisting of triethanolamine, NaOH, KOH, and sodium bicarbonate. Other biocompatible pH modifiers are envisioned that are suitable for use in humans, and the disclosure is not intended to be limited by the type of pH modifier used.

The method according to the disclosure can be optimally performed with a kit. In certain embodiments, the kit comprises vessels, which can vary greatly in the volume of components they can hold. In some embodiments, the vessels are vials that can hold no more than 5 ml. In other embodiments, the vessels can hold multiple liters or even larger industrial volumes are envisioned. The disclosure is not limited by the volume of the vessels or vials. For the sake of making the disclosure simpler, reference to vials includes reference to vessels and vice versa.

In some embodiments, the kit comprises at least one first vial comprising lyophilized protein mix, at least one second vial comprising diluent mix, at least one third vial comprising initiator mix, and at least one first syringe, preferably a Luer-lock syringe. With the kit, step 1 and step 1(a) can be performed by injecting a first syringe filled with diluent mix obtain from second vial into the first vial comprising lyophilized protein mix to produce a precursor solution. The first vial can then be mechanically mixed to obtain a homogeneous precursor solution. The precursor solution can then be pulled back into the first syringe.

Step 2 can be performed by pulling initiator mix from the third vial into a second syringe and connecting the first syringe and the second syringe and mechanically mixing their contents to form a hydrogel.

In some embodiments, a diluent mix is added to a vessel comprising lyophilized protein mix to form a precursor solution which and can then be homogenized. A volume of initiator can be added to the precursor solution to form a hydrogel having a homogenized bioactive protein mix. The broad-spectrum supplement composition can include components of the lyophilized protein mix. It can also include other components described herein that might or might not be part of the lyophilized protein mix and can be included at various stages in various additional mixing steps. For example, additives can be included in the precursor solution. Additionally, more precursor solution with dissolved additives might be added after formation of the hydrogel to incorporate additional additives or constituents, or to modify the properties of the hydrogel.

The disclosure further relates to a hydrogel composition comprising a hydrogel and a broad-spectrum supplement composition. It is envisioned that the broad-spectrum supplement composition can contain a plurality of anti-aging and skin-rejuvenating agents, compounds, peptides, growth factors, and additives. In certain embodiments, the broad-spectrum supplement composition comprises at least one growth factor selected from the group consisting of bFGF, EGF, GDF-11, HGF, KGF, PDGF-AA, TGF-b1, and VEGF. In other embodiments, the hydrogel further comprises PLGA nanospheres, wherein the growth factors can be adsorbed onto the nanospheres for sustained release from the hydrogel. In some preferred embodiments, the PLGA nanospheres are approximately 100 to 300 nm.

It is envisioned that the hydrogel composition can further include micro-spicules which can be massaged into the skin to promote skin penetration of the PLGA nanospheres. In other embodiments, cell penetrating peptide linkers are bound to the growth factors to promote skin permeation and cell penetration.

The benefit of producing the hydrogel at the time of use allows the proteins to remain freeze-dried “fresh” and undegraded until use. In addition, it allows for thorough mixing of the diluent then removal of the mixture to the application syringe/vessel before initiating a viscosity change which promotes homogeneity of the mixture. Due to the fragile nature of proteins, the difficulty of removing gels from mixing containers, and the need for thorough mixing of the gel mixture, the present method and composition presents an improvement over previously known methods for the preparation and application of cosmetic hydrogels because it allows for fresh reconstitution of fragile bio-products and better homogeneity prior to formation of the hydrogel.

In certain embodiments, the optimum acrylate/carbomer percent is approximately 0.5% and a pH initiator strength of at least 50% and 1% of the entire diluent/carbomer amount. A lower percentage of carbomer may not gel sufficiently when the initiator is introduced at one end of the vessel.

In certain embodiments, the diluent can comprise water, viscosity modifying agent (carbomer), optionally an amino acid protein stabilizer, optionally a humectant, optionally a preservative, optionally an emollient, optionally anti-aging ingredients. In certain embodiments, these components can be found in the precursor solution being added to the lyophilized proteins. In other embodiments, a diluent or precursor solution can optionally contain water, viscosity modifying agent (carbomer), optionally an amino acid protein stabilizer, optionally a humectant, optionally a preservative, optionally proteins, optionally peptides, optionally an emollient, optionally anti-aging ingredients for adding in a later step such as after hydrogel formation and can serve to further modify the properties of the hydrogel.

In certain embodiments, the amino acid protein stabilizer can be glycine. It is also envisioned that the amino acid protein stabilizer can be in an amount preferably of 5 mg/ml to 30 mg/ml. In other embodiments, the viscosity modifying agent can be: a polyacrylic acid (carbomer). In certain embodiments, the viscosity modifying agent is present from 0.1 to 1%, most preferably 0.2% to 0.5%. It is also envisioned that the viscosity modifying agent can be in low viscosity unneutralized form. In certain embodiments, the state change inducer/initiator can be a pH modifier, a photoinitiator, or a free radical initiator. It is also envisioned that the initiator is introduced at a future time. In certain embodiments, the initiator is introduced at a percentage comprising 0.01 to 5% preferably 1%. It is also envisioned that the inducer/initiator can be triethanolamine, NaOH, KOH, sodium bicarbonate.

In certain embodiments, light can be used to initialize polymerization of the hydrogel in just one syringe or vessel instead of the chemical process which requires two syringes or vessels. In some embodiments, UV-initiated polymerization of the hydrogel involves the formation of a crosslinked polymer network by using a UV-sensitive monomer or a mixture of monomers, which are capable of undergoing polymerization upon exposure to UV light.

The process typically starts with the mixing of the monomers with a crosslinking agent and a photoinitiator, which is a chemical compound that absorbs UV light and initiates the polymerization process. The mixture is then exposed to UV light, which triggers the polymerization process.

In some embodiments, light-initiated polymerization of a hydrogel involves the use of a photosensitive monomer or a mixture of monomers that are capable of undergoing polymerization upon exposure to light. The process is similar to UV-initiated polymerization, but it uses visible light or other forms of radiation instead of UV light.

The process starts with the mixing of the monomers with a crosslinking agent and a photoinitiator that is sensitive to the wavelength of light used for polymerization. The mixture is then exposed to the light source, which initiates the polymerization process.

During the polymerization process, the photoinitiator absorbs the light and undergoes a chemical reaction that generates free radicals. These free radicals then react with the monomers, leading to the formation of polymer chains. The crosslinking agent is also incorporated into the growing polymer chains, forming a three-dimensional network structure.

The resulting hydrogel has a high water content and can exhibit various properties such as biocompatibility, mechanical strength, and responsiveness to environmental stimuli such as temperature, pH, and ionic strength

A hydrogel according to the disclosure can be made according to the methods in the following examples. The disclosure is not intended to be bound by the concentrations, volumes, and weights given as other concentrations, volumes, and weights may suffice to obtain a hydrogel according to the unique method disclosed herein and having the superior and unexpected synergistic properties of the presently disclosed hydrogel, though certain ratios are considered optimal and preferable.

The present disclosure relates to a method and kit for diluting a lyophilized protein mix with a special viscosity blended constitution fluid by injecting said constitution fluid through an injection port of an atomizer cap, thereby creating a spray solution.

In certain embodiments, the lyophilized protein mix or the constitution fluid can further comprise at least one additive selected from the group consisting of amino acid stabilizer, humectant, preservative, emollient, and anti-aging ingredients. These additives can not only provide stabilization of the peptides and preservation of the spray solution, but they can also include additional anti-aging ingredients and other active components that promote synergistic effects when combined with growth factor peptides.

In certain embodiments, the amino acid stabilizer is glycine, however, other amino acid stabilizers are envisioned and suitable and the disclosure is not intended to be limited by the type of amino acid stabilizer. In some embodiments, the amino acid stabilizer is included in the solution in a preferable concentration range of between 5 mg/ml to 30 mg/ml.

The method according to the disclosure can be optimally performed with a kit comprising at least one vial comprising lyophilized constituents and at least one syringe comprising constitution fluid. The kit further comprises a uniquely designed atomizer cap attached to the vial of lyophilized constituents, the atomizer cap comprising a syringe injection port disposed through a top of the atomizer lid, whereby a customer can deliver the constitution fluid in a clean and scaled environment to activate the lyophilized constituents inside.

The disclosure further relates to a spray solution composition comprising constitution fluid and a broad-spectrum protein composition. It is envisioned that the broad-spectrum protein composition can contain a plurality of anti-aging and skin-rejuvenating agents, compounds, peptides, growth factors, and additives. In certain embodiments, the broad-spectrum protein composition comprises at least one growth factor selected from the group consisting of bFGF, EGF, GDF-11, HGF, KGF, PDGF-AA, TGF-b1, and VEGF. In other embodiments, the spray solution further comprises PLGA nanospheres, wherein the growth factors can be adsorbed onto the nanospheres for sustained release from the spray solution. In some preferred embodiments, the PLGA nanospheres are approximately 100 to 300 nm.

It is envisioned that the spray solution composition can further include micro-spicules which can be massaged into the skin to promote skin penetration of the PLGA nanospheres. In other embodiments, cell penetrating peptide linkers are bound to the growth factors to promote skin permeation and cell penetration.

The benefit of producing the spray solution at the time of use allows the proteins to remain freeze-dried “fresh” until use. Due to the fragile nature of proteins and the difficult constraints of shipping and handling these components, the present method and composition presents an improvement over previously known methods for the preparation and application of cosmetic spray solutions. The lyophilized protein mix is mixed with a fluorescent protein that glows under UV black light. If the fluorescent protein fails to glow under UV light or the glow diminishes, then this is an indication that the other enzymes, peptides, and proteins in the mixture have also degraded.

In certain embodiments, the constitution fluid consists of water, viscosity modifying agent (carbomer), optionally an amino acid protein stabilizer, optionally a humectant, optionally a preservative, optionally an emollient, optionally anti-aging ingredients.

In certain embodiments, the amino acid protein stabilizer can be glycine. It is also envisioned that the amino acid protein stabilizer can be in an amount preferably of 5 mg/ml to 30 mg/ml. In other embodiments, the viscosity modifying agent can be: a polyacrylic acid (carbomer). In certain embodiments, the viscosity modifying agent is present from 0.1 to 1%, most preferably 0.2% to 0.5%. It is also envisioned that the viscosity modifying agent can be in low viscosity unneutralized form. In certain embodiments, a state change inducer/initiator can be included and can be a pH modifier, a photoinitiator, or a free radical initiator. It is also envisioned that the initiator can be introduced at a future time. In certain embodiments, the initiator r is introduced at a percentage comprising 0.01 to 5% preferably 1%. It is also envisioned that the inducer/initiator can be triethanolamine, NaOH, KOH, sodium bicarbonate.

A spray solution according to the disclosure can be made according to the methods in the following examples. The disclosure is not intended to be bound by the concentrations, volumes, and weights given as other concentrations, volumes, and weights may suffice to obtain a spray solution according to the unique method disclosed herein and having the superior and unexpected synergistic properties of the presently disclosed spray solution, though certain ratios are considered optimal and preferable.

### EXAMPLE 1

Protein mix: To 0.9 liter of deionized water chilled to 4° C. placed in a 2-liter polypropylene Erlenmeyer flask was subsequently added and magnetically stirred until dissolved: 999 g monobasic sodium phosphate, 999 g dibasic sodium phosphate, 50 g trehalose, 1 g bovine serum albumin, 25 ug of recombinantly produced and purified human sequence bioidentical growth factors. QS Water was added to make 1 liter and this mixture was subsequently filtered and sterilized by 0.2-micron filtration.

Constitution Fluid Mix: To 0.9 liter of deionized water placed in a 2-liter polypropylene Erlenmeyer flask and magnetically stirred until dissolved and homogenous: 1.46 g (5 mM) EDTA, 30 g (30 mg/ml) glycine, 5 g (or less) Carbomer 940 QS water to make 1 liter and then 0.2-micron filtered.

Initiator Mix: To 0.5 liter of deionized water placed in a 2-liter polypropylene Erlenmeyer flask and magnetically stirred until homogenous: 0.5 liter ethylenediamine. Subsequently, the mixture was 0.2-micron filtered.

Lyophilized Protein Mix: The aforementioned protein mix above was filled into 1-3 ml vials that were positioned in aluminum heat-sink trays and filled by a peristaltic pump set to a quantity of 1 ml per vial. Subsequently, the trays were loaded into a 48 cubic foot freezer and frozen to approximately −30° C. After reaching equilibrium, the trays were loaded into a pre-cooled freeze dryer and lyophilized for approximately 24 hours. Vials were removed from the freeze dryer and placed into an automatic stoppering and capping machine for sealing. Finally, lyophilized vials were stored at −30° C.

Reconstitution: A glass Luer-lock syringe filled with 1 ml of constitution fluid was injected into the Lyophilized protein mix vial through an injection port on the top of an atomizer cap of the Lyophilized protein mix vial creating a spray solution, and the spray solution was rolled for 10 seconds for homogenization. Contents are in spray solution form at this point and are easily applied to epidermal areas. In some embodiments, the Initiator Mix can be injected through the injection port to induce desired viscosity or other changes to the spray solution.

Alternate Reconstitution: To a 100 ml Boston round glass container containing the lyophilized protein mix was added the constitution fluid and the container was capped with an atomizer and rolled end-over-end for 15 seconds to gently mix contents. Subsequently, an initiator mix can be added to affect viscosity or other properties of the spray solution.

### EXPERIMENT 2

Protein mix: To 0.9 liter of deionized water chilled to 4° C. placed in a 2-liter polypropylene Erlenmeyer flask was subsequently added and magnetically stirred until dissolved: 999 g monobasic sodium phosphate, 999 g dibasic sodium phosphate, 50 g trehalose, 1 g bovine serum albumin, 25 ug of recombinantly produced and purified human sequence bioidentical growth factors. QS Water was added to make 1 liter and this mixture was subsequently filtered and sterilized by 0.2-micron filtration.

Constitution Fluid Mix: To 0.9 liter of deionized water placed in a 2-liter polypropylene Erlenmeyer flask and magnetically stirred until dissolved and homogenous: 1.46 g (5 mM) EDTA, 30 g (30 mg/ml) glycine, 5 g (or less) Carbomer 940 QS water to make 1 liter and then 0.2-micron filtered.

Initiator Mix: To 0.5 liter of deionized water placed in a 2-liter polypropylene Erlenmeyer flask and magnetically stirred until homogenous: 0.5 liter ethylenediamine. Subsequently, the mixture was 0.2-micron filtered.

Lyophilized Protein Mix: The aforementioned protein mix above was filled into 1-3 ml vials that were positioned in aluminum heat-sink trays and filled by a peristaltic pump set to a quantity of 1 ml per vial. Subsequently, the trays were loaded into a 48 cubic foot freezer and frozen to approximately −30° C. After reaching equilibrium, the trays were loaded into a pre-cooled freeze dryer and lyophilized for approximately 24 hours. Vials were removed from the freeze dryer and placed into an automatic stoppering and capping machine for sealing. Finally, lyophilized vials were stored at −30° C.

Reconstitution: A glass Luer-lock syringe filled with 1 ml of constitution fluid was injected into the Lyophilized protein mix vial through an injection port on the top of an atomizer cap of the Lyophilized protein mix vial creating a spray solution, and the spray solution was rolled for 10 seconds for homogenization. Contents are in spray solution form at this point and are easily applied to epidermal areas. In some embodiments, the Initiator Mix can be injected through the injection port to induce desired viscosity or other changes to the spray solution.

Alternate Reconstitution: To a 100 ml Boston round glass container containing the lyophilized protein mix was added the constitution fluid and the container was capped with an atomizer and rolled end-over-end for 15 seconds to gently mix contents. Subsequently, an initiator mix can be added to affect viscosity or other properties of the spray solution.

### Anti-Aging Protein Mix

The present disclosure relates to an anti-aging growth factor blend in a defined synergistic ratio, and a method for solubility enhancement/protease inhibition combined with a skin penetration peptide via bioconjugation.

There is a synergy between anti-aging and repair growth factors to decrease overall dosage and obtain a more significant effect with a lower amount, and increasing the safety profile.

In certain embodiments, the broad-spectrum supplement composition can include a protein mix comprising at least one of a plurality of growth factors including bFGF, EGF, GDF-11, HGF, KGF, PDGF-AA, TGF-b1, and VEGF. In certain embodiments, the protein mix is present in a final hydrogel in a concentration of less than 1 mg/ml. According to certain embodiments with said improved synergistic effect, the formulation for synergy and approximate ratios are as follows:

TABLE 1
Ratio of Active Ingredients for synergistic effect

| Active | | lyophilized amount mixed into |
|---|---|---|
| Ingredients | Ratio | approximately 1 to 2 ml |
| bFGF | 250.00 | 2.634 ng- |
| | | 263.4 ng |
| EGF | 250.00 | 2.306 ng- |
| | | 2305.72 ng |
| GDF-11 | 5.00 | 50 pg- |
| | | 5 ng |
| HGF | 20.00 | 191 pg- |
| | | 19.142 ng |
| KGF | 5.00 | 45 pg- |
| | | 4.5 ng |
| PDGF-AA | 20.00 | 192 pg to |
| | | 19.206 ng |
| TGF-b1 | 10.00 | 90 pg- |
| | | 9.025 ng |
| VEGF | 7.00 | 69 pg- |
| | | 6.906 ng |

These growth factor proteins coupled with a polyethylene glycol linker can be attached at various amino sites linked to a cell-penetrating peptide. This growth factor complex builds skin complete in dermal profile. The growth factor complex peptides promote the creation of blood vessels and skin elements. The effect is new skin capable of being in a more youthful state particularly due to increased hyaluronic acid and collagen synthesis. Reducing wrinkles and maintaining a more hydrated state allows for faster removal of damaging free radicals.

The ability to have the growth factor complex penetrate the skin topically allows for continual administration which is substantially better at reinforcing the messaging capability of the growth factors and causing their beneficial effects.

The synergistic combination of growth factors in the complete growth factor complex allows for lower, more tolerable dosages of anti-aging, skin rejuvenating proteins, avoiding the addition of negative proteins and higher concentrations that can cause inflammation, like are present in existing topical stem cells treatments. Additionally, existing stem cell treatments are inherently flawed because they require injection of live stem cells daily to achieve optimal results as constant administration is how cell communication works naturally. Most people do not have the equipment, means, or knowledge to maintain viable stem cell lines. Stem cells constantly die in vitro and require proper storage and handling. The hydrogel and spray solution of the present disclosure avoids these problems by relying on lyophilized stem-cell derived growth factors, proteins, peptides, and other supplements, which are not reconstituted until just prior to treatment, thereby protecting these active ingredients from degradation prior to use.

Another embodiment of this invention is the topical adsorption of the lyophilized growth factor complex with or without cell-penetrating peptide/linker attached to the surface of PLGA (polylactic glycol acid) nanospheres of approximately 100 to 300 nm for sustained release application admixed into a hydrogel. Once the Growth Factor Complex is adsorbed onto the nanospheres and a micro tunnel via micro-needling or sponge spicule created micro tunnel is created, the spheres can be massaged into the face allowing sustained release.

### CPP Linker Attached Growth Factor Synthesis

To 87 μg Protein-SH was added 9 μg of NHS-PEG12-Maleimide (succinimide-[(N-maleimidopropionamido)-dodecaethyleneglycol] ester) in previously dried over molecular sieves DMSO. To 24.82 μg in 36.70 μl of the above ratio of growth factors were subjected to conjugation procedure.

After incubating the reaction mixture for 2 hours at 4° C. with slow agitation the excess conjugation crosslinker was removed with a 0.5 ml Zeba spin column that was previously equilibrated with conjugation buffer PBS.

### Fluorescent Protein

As another feature of the present disclosure, a fluorescent protein is included in the lyophilized protein mix for enabling determination of enzymatic activity and viability of bioactive ingredients of the lyophilized protein mix, for example, lyophilized protein mix dissolved in precursor solution, hydrogel, or components of the spray solution. In one embodiment, it is envisioned that the spray solution can include a modified fluorescent protein in sufficient concentration to fluoresce upon application of a frequency of light, such as, in some embodiments, UV light or black light.

In some embodiments, the lyophilized protein mix includes a fluorescent indicator such as GFP or BFP—although other fluorescent proteins are envisioned and the disclosure is not limited by the type of fluorescent protein, as other fluorescent proteins and indicators are envisioned. Additionally, the disclosure is not limited to UV-light or black light or any particular light frequency. Other suitable frequencies of light which induce fluorescence of fluorescent proteins and indicators are envisioned.

After constituting the freeze-dried proteins into the spray solution, precursor solution, or hydrogel, a UV light specially can induce a fluorescence of the specific concentration of the modified fluorescent protein. If the product glows at a certain level upon shining the UV light on the spray solution, precursory solution, or hydrogel, the customer will know that the product is contains fresh, bioactive, and ready to use proteins, peptides, enzymes, and other peptide constituents in the lyophilized protein mix. The level of fluorescence can be used to predict a corresponding enzymatic activity and viability of bioactive ingredients. Failure to glow or a diminished glow upon application of UV light would be an indication that the bioactive ingredients have de-natured and are less viable as compared to a fresh product. Other means for determining the enzymatic activity and viability of bioactive ingredients are also envisioned and considered part of the disclosure.

### Components According to Some Embodiments Shown in the Figures

A kit 100 for making a hydrogel 101 is disclosed. The kit 100 comprises at least one vessel 102 containing lyophilized protein mix 103, at least one vessel containing diluent mix 104, and at least one vessel containing initiator mix 105. The lyophilized protein mix comprises at least one protein. The protein maybe selected from the growth factors including bFGF, EGF, GDF-11, HGF, KGF, PDGF-AA, TGF-b1, and VEGF, disclosed herein, as well as other growth factors. In some embodiments, the lyophilized protein mix includes fluorescent proteins 113 which fluoresce under black light conditions thereby demonstrating the viability of the lyophilized protein mix 103. In some embodiments, the vessels 102 are vials 106. The vessels can be capped with a microfilter 116 to keep out contaminants. The diluent mix is 117 and the initiator mix is 118 in the figures. Additionally, the kit 100 may further include a syringe 114 with a Luer-Lock 115. In some embodiments, it includes a first Luer-lock syringe 107, a second Luer-lock syringe 108, a syringe-to-syringe connector 109 to connect syringes, a vial-to-syringe adaptor 110 to connect vials with syringes, and a black light 111 to illuminate fluorescent protein. In certain embodiments, the vial containing lyophilized protein mix is 112, the vial containing diluent mix is 104, and the third vial containing initiator mix is 105. Furthermore, the kit 100 may also comprise twenty-nine vials containing lyophilized protein mix 112, three vials containing diluent mix 104, and three vials containing initiator mix 105.

1. A method of testing viability of a reconstituted lyophilized protein mix, comprising:
reconstituting lyophilized protein mix into a diluent mix to make a precursor solution in a vessel, the lyophilized protein mix comprising a fluorescent protein and at least one protein, the diluent mix comprising water;
testing viability of reconstituted lyophilized protein mix by shining a light upon the reconstituted lyophilized protein mix of the vessel; wherein,
a fluorescence of the vessel under light demonstrates viability of the lyophilized protein mix; and wherein,
a lack of fluorescence or a diminished fluorescence of the vessel indicates diminished viability of the lyophilized protein mix.

2. The method according to claim 1 further comprising:
adding an initiator mix to the precursor solution to make a hydrogel, the initiator mix inducing an increase in viscosity.

3. The method according to any of claims 1-2, wherein the diluent mix comprises a carbomer.

4. The method of any of claims 1-2, wherein the precursor solution further comprises an amino acid stabilizer, humectant, preservative, emollient, or an anti-aging molecule, compound, protein, or peptide.

5. The method of any of claims 1-2, wherein the precursor solution further comprises an amino acid stabilizer.

6. The method of any of claims 1-2, wherein the precursor solution further comprises a humectant.

7. The method of any of claims 1-2, wherein the precursor solution further comprises a preservative.

8. The method of any of claims 1-2, wherein the precursor solution further comprises an emollient.

9. The method of any of claims 1-2, wherein the precursor solution further comprises an anti-aging molecule, compound, protein, or peptide.

10. The method of claim 2, wherein the initiator mix is in low viscosity, unneutralized form.

11. The method of claim 2, wherein the initiator mix comprises a pH modifier, a photoinitiator, or a free radical initiator.

12. The method of claim 2, wherein the initiator mix comprises a pH modifier.

13. The method of claim 2, wherein the initiator mix comprises a photoinitiator.

14. The method of claim 2, wherein the initiator mix comprises a free radical initiator.

15. The method of claim 2, wherein the initiator mix is introduced at a percentage of 0.01 to 5% of total solution.

16. The method of claim 2, wherein the initiator mix is introduced at a percentage of 1% total solution.

17. The method of claim 2, wherein the initiator mix comprises at least one pH modifier selected from the group consisting of triethanolamine, NaOH, KOH, and sodium bicarbonate.

18. The method of any of claims 1-2 wherein:
diluent mix is added into a vessel comprising lyophilized protein mix making a precursor solution;
adding initiator mix to the precursor solution to form a hydrogel.

19. The method of any of claims 1-3, wherein, wherein the carbomer is present from 0.1 to 1% hydrogel volume.

20. The method of any of claims 1-3, wherein the carbomer is present in the hydrogel from 0.2% to 0.5% hydrogel volume.

21. The method of any of claims 1-3, wherein the hydrogel comprises a carbomer at a percentage of approximately 0.5% total solution; and a pH initiator at 1% hydrogel volume.

22. The method of any of claims 1-5, wherein the amino acid stabilizer is glycine.

23. The method of any of claims 1-5, wherein the amino acid stabilizer is included in the hydrogel in a preferable concentration of between 5 mg/ml to 30 mg/ml.

24. A precursor solution comprising:
a lyophilized protein mix dissolved in a diluent, the protein mix comprising at least one protein and at least one fluorescent protein which fluoresces under light.
